(19) United States
(12) Patent Application Publication
(10) Pub. No.: US 2012/0171742 A1
(43) Pub. Date: Jul. 5, 2012

(54) Method and Apparatus for Treating Fermentable Substances

(75) Inventors: Seine Roelofs, CB Bakel (NL); W.J.H. Kuster, CN Nijmegen (NL)

(73) Assignees: JWF BEHEER BV, CN Nijmegen (NL); ROMIL BEHEER BV, CB Bakel (NL)

(21) Appl. No.: 13/394,339

(22) PCT Filed: Sep. 6, 2010
(86) PCT No.: PCT/EP2010/063015

§ 371 Date: Mar. 6, 2012

(30) Foreign Application Priority Data
Sep. 7, 2009 (DE) 10 2009 040 195.4

(51) Int. Cl. C12P 5/02; C12M 1/02

(52) U.S. Cl. 435/167; 435/289.1

(57) ABSTRACT

The present invention relates to a method for treating fermentable organic substances, the method comprising the anaerobic fermentation thereof in a fermentation tank (10), and to equipment which is used to mix the fermentation material and is located, at least in part, outside the fermentation tank. In the case of the method according to the invention, the fresh fermentation material fed, in the first instance, is fed to a mixing tank (14), which extends preferably annularly around the fermentation tank; in this mixing tank, the fermentation material is conveyed over an annular path, mixed into a mash and, after a certain residence time, conveyed out of the mixing tank, and fed to the fermentation tank, by means of a vacuum pressure pumping system (20). The quantity of fermentation material which is removed from the mixing tank in each case can be measured if the tank used for this purpose is standing on weighing cells. The invention provides a method and an apparatus which, for treating organic substances, allow effective mixing of the fermentation material in the phase prior to the latter being fed to the cylindrical tank or fermentation tank, better conveying of the fermentation material and better control of the fermentation process.

### CROSS REFERENCE TO RELATED APPLICATIONS

This application is the U.S. national stage of International Application No. PCT/EP2010/063015, filed on Sep. 6, 2010, and claims the benefit thereof. The international application claims the benefits of German Application No. 102009040195.4 filed on Sep. 7, 2009; all applications are incorporated by reference herein in their entirety.

### BACKGROUND

The present invention relates to a method for treating fermentable organic substances, the method comprising the anaerobic fermentation thereof in at least one fermentation tank, and to equipment which is used to mix the fermentation material, wherein the fresh fermentation material fed, in the first instance, is fed to a mixing tank in which the fermentation material is chopped up if necessary, mixed into a thick-flowing mash, and only thereafter is this mixture fed to a fermentation tank for further fermentation. The present invention further relates to apparatus for use in a method of the type described above.

Because of the impending depletion, expected at some time in the future, of the natural resources of crude oil and natural gas, there is a search for new energy sources that can be used permanently. In this context, in particular, energy sources are of interest that have a reduced hazard potential when compared to nuclear power, and which can be used without having a damaging effect on the climate. One of the known options, the use of hydrogen, is associated with a disadvantage in that hydrogen is a difficult-to-handle highly-explosive gas when exposed to air, and consequently this technology has a very considerable hazard potential. Other known methods for using alternative energy sources in the form of so-called renewable raw materials, for example the extraction of rapeseed oil methyl ester from rapeseed cultivation, are associated with a disadvantage in that they produce a comparatively low yield per hectare of cultivation, and are thus not particularly suited to completely replace fossil fuels.

One of the methods for obtaining energy from alternative sources, namely the production of biogas (essentially methane) from biomass, has become more and more important over recent years. In this process biogas is obtained, by means of microorganisms, by anaerobic fermentation of biomass of various origin. For example liquid manure can be used as biomass, with liquid manure being available in large quantities in agricultural operations involving cattle. As an alternative it is also possible to process, for example, sewage sludge, dung, biological waste, food scraps, or biomass from energy plants that have been cultivated expressly for this purpose. Moreover, other organic waste of any kind can be mixed into the biomass. As a rule, the biogas is subsequently used in power generation and the power is fed to the electricity grid. As an alternative, biogas is presently also to an increasing extent fed to the public gas network after gas treatment (methane enrichment). Treatment of the fermentation material usually takes place in sizeable tanks where first of all hydrolysis takes place during which the biopolymers are enzymatically decomposed into smaller components such as fatty acids, sugars, amino acids, which then during the subsequent fermentation are transformed by anaerobic bacteria to form weak organic acids and finally acetic acid. In the further transformation methane then arises from the acetic acid.

For mixing the fermentation medium in fermentation tanks for biogas production, according to the state of the art predominantly agitators are presently used, for example submerged agitators. Less frequently, as described in DE 37 37 870 A 1, plate-shaped mixing bodies are used, which are mechanically moved up and down in the tank.

In DE 10 2007 024 947 A1 a biogas fermenter is described in which an axial agitator for circulating the biomass in the digester is arranged outside the actual fermenter. In this arrangement biomass from the digester is sucked into circulation pipes, is mixed by an agitator arranged in the pipe, and is subsequently pumped back to the fermenter. It should be pointed out that the use of agitators that are arranged within the digester itself can be disadvantageous. For example, in the case of a defect it would be necessary to empty the entire digester. Moreover, the inhomogeneity and tenacity of the biomass present in the digester creates problems during circulation by means of conventional agitators. The solution described in the above-mentioned document proposes that the agitator be moved to the region outside the digester. However, since the biomass from the digester is sucked into circulation pipes, and is also subsequently pumped back to the digester by way of pipes, fluidic obstructions occur as a result of the comparatively small cross sections of these pipes. Furthermore, as a result of the design, the proportion of biomass that is being mixed at a given time is comparatively small relative to the entire volume of the biomass in the tank. In this known plant new biomass, too, is fed to the fermentation tank by way of a tubular feeder, and consequently corresponding limits result.

In DE 10 2007 005 069 A 1 a biogas plant is described in which several annular fermentation tanks are concentrically arranged inside each other. However, in this known biogas plant the main fermenter is situated concentrically in the interior, and the fermentation material subsequently reaches a radially-outward positioned annular channel tank that is used as a postfermenter. The tanks are separated from each other by intermediate ring walls. However, the fermentation material, by overflowing over the intermediate ring wall, flows from the open-top main fermenter directly into the postfermenter, or is pumped from one tank to the other by means of a pump arranged above the tank. In this arrangement the tanks comprise a shared gas reservoir above the tank compartments that are separated from each other only by intermediate walls. In this known biogas plant no mixing tank in the sense of the present invention is used. In particular, no mixing process is provided in which hydrolysis of the fermentation material takes place. Instead, all the tank compartments are used for biogas production; in other words the actual fermentation process takes place in them. The fermentation material is pumped within the tank from the tank compartment of the main fermenter to that of the postfermenter; however, said fermentation material is not pumped from the tank between individual fermentation stages. In this known biogas plant the use of the tank compartments that are arranged annularly within each other takes place because of the desire to create a shared gas storage device above the separate tank compartments.

From DE 10 2005 054 323 A1 a further biogas plant is known, in which the fermentation chamber of a main fermenter in which biogas production takes place extends in an annular shape around a settling chamber. The fermentation material is pumped directly from the fermentation chamber to the settling chamber. In the fermentation chamber predominantly biogas production takes place, with the fermentation material only reaching the settling chamber after it has to a large extent fully fermented. However, both tanks are of an open-top design and are interconnected by way of a shared gas bell. In this arrangement the settling chamber comprises a fixed-bed reactor and has the task of returning, by overflowing, to the fermentation tank any active biomass still present in the fermentation material, thus recovering said biomass. This embodiment, too, does not provide a mixing tank in the sense of the present invention.

DE 197 46 636 A1 describes a method of the type mentioned in the introduction, comprising a biogas plant in which within a reactor housing there is a smaller mixing tank, fermentation tank and initial supply tank in which the fresh organic material is subjected to anaerobic fermentation and is shredded by means of a helical circulator. By way of a feeder arranged in the reactor housing, this pre-acidified organic material is then conveyed to a cylindrical reaction vessel located in the centre of the reactor housing, which reaction vessel comprises a heater, so that in this reaction vessel the organic material can be heated. From the reaction vessel the essentially already fermented material reaches an intermediate region that extends concentrically around the reaction vessel. However, in this intermediate region only post-gasification and storage of the previously treated material takes place; in other words the reaction vessel can be considered to be the main fermentation tank. In this known plant, too, the material is conveyed from the smaller mixing tank directly to the reaction vessel, and likewise it is conveyed from the reaction vessel directly to the intermediate region.

In DE 199 28 663 A1 a method for treating biological waste is described, in which method at first the fermentation material is shredded and subsequently, with the addition of water, is placed in a separate cylindrical hydrolysis tank where it is circulated and homogenised by means of an agitator. However, in this method aerobic hydrolysis is aimed for, and for this purpose a quantity of air is blown into the fermentation material. From the hydrolysis tank the fermentation material is pumped to a receiver tank in which there is constant agitation. From this receiver tank a paste-like material is pumped into a special disc reactor that comprises a horizontal pipe comprising a motor-driven shaft with perforated discs, wherein biogas production takes place in this disc reactor.

In DE 195 38 579 C1 a biogas plant is described which comprises an outer tank and an inner tank that is concentrically arranged within the aforesaid, wherein in addition a separating wall extending in radial direction is provided between the two tanks. However, the two tanks comprise a shared gas space above the level of liquid manure, and by way of corresponding devices, for example a circulation pipe and an overflow, direct conveyance of the fermentation material from the inner to the outer tank and vice versa for the purpose of mixing is provided. There is thus no mixing tank in the sense of the present invention, in which mixing tank a separate and different fermentation process takes place, nor are there any provisions for controlling the residence time of the fermentation material in one tank independently from the processes taking place in the other tank.

From the German patent specification DE 10 96 754 C it is known, in principle, to use a vacuum pressure pump for conveying media comprising solids, which media includes, for example, liquid manure, by means of which vacuum pressure pump the medium can be sucked in, filled into a tank, and pushed from the tank with the application of pressure. However, the above-mentioned printed specification relates to liquid manure tankers by means of which tankers liquid manure can be spread onto fields for the purpose of fertilising; said printed specification does not contain any suggestion for the use of such pumping devices in the context of biogas plants.

In a biogas plant it is advantageous to produce already in the mixing phase a comparatively thick-flowing energy-rich mash from the fermentation material. However, such a thick-flowing mash is difficult to pump. Therefore the use of pipes with limited cross sections is just as disadvantageous as are excessively long pumping distances, because the thick-flowing fermentation material must then be aspirated over longer distances.

### SUMMARY

This is where the present invention applies. It is the object of the invention to provide a method or apparatus for treating organic substances of the type stated in the introduction, which method and/or apparatus with constructively simple technical means makes possible effective mixing of the organic substances or of the fermentation material already in the phase prior to feeding it to the cylindrical tank or fermentation tank. Furthermore, it is the aim to feed to the fermentation tank a medium that is as thick-flowing and homogeneous as possible, with the lowest-possible water content, in order to in this manner increase the yield during biogas production.

This object is met by a method of the type mentioned in the introduction with the characteristics of the main claim, or by apparatus with the characteristics of the device-related claim 9.

According to the invention it is proposed that after a certain residence time the fermentation material is conveyed out of the mixing tank and fed to the fermentation tank by means of a vacuum pressure pumping system.

The organic products to be treated are thus in the first instance fed to the annular mixing tank that is separated from the actual tank, in particular the fermentation tank, by a wall, which arrangement provides an advantage in that the fermentation process in the main tank takes place independently of the process of mixing. Likewise, the gas space above the fermentation tank is completely separate from the gas space of the mixing tank. The mixing tank can, for example, have a smaller holding volume, in particular a significantly smaller holding volume, than the main (fermentation) tank. The new material to be treated is effectively mixed in this mixing tank and is only fed to the main tank (fermentation tank) after a defined period of time. This provides an advantage in that the residence time in the mixing tank can be selected and varied as required. The transformation processes taking place in the mixing tank proceed completely independently of the fermentation processes in the fermentation tank. In the solution according to the invention it is not at all the case that the addition of a particular quantity of fresh medium added to the mixing tank results in a corresponding quantity of fermentation material being conveyed out of the mixing tank into the fermentation tank, as is the case in known systems that comprise intermediate walls between the two tanks, by way of which intermediate walls the fermentation material can overflow. The method according to the invention is thus considerably more flexible. It is thus possible, for example, to remove smaller quantities of fermentation material from the mixing tank than were previously fed to it, for example if one wishes to increase the residence time in the mixing tank. It is also possible to admix further substances to the fermentation material removed from the mixing tank, before the fermentation material is pumped to the fermentation tank. Uncontrolled mixing of the medium in the mixing tank with the material in the fermentation tank is avoided. The method according to the invention provides the option in the first instance of checking the consistency of the fermentation material removed from the mixing tank, and, if necessary, to decide to implement a longer residence time, or to add further substances.

### DETAILED DESCRIPTION

There is a further advantage if, according to a preferred improvement of the method according to the invention, the weight of the substance quantity removed from the mixing tank and/or the substance quantity fed to the fermentation tank is acquired, for example, with the use of a vacuum pressure pump comprising a tank that is positioned on a weighing device by means of which the weight of the respective substance quantity placed in the tank and/or of the substance quantity removed from said tank are/is acquirable. The quantities acquired in this process can be recorded or stored in a computer so that subsequent evaluation becomes possible. This makes it possible to achieve more precise control and optimisation of the method-related procedures.

The above-mentioned acquisition of the quantity of fermentation material in each case fed to the fermentation tank provides yet other advantages. There are many biogas plants with a movable (interior) gas roof that moves upwards as the gas quantity in the tank increases. According to a preferred improvement the present invention provides for a slight reduction of the quantity of fermentation material fed to the fermentation tank per unit of time, for example if a rise in the gas roof is detected, which signals an increased quantity of gas in the fermentation tank. If a lowering of the gas roof is detected, it is possible to increase the fed quantity of fermentation material. The supply of the respective quantity of fermentation material, or the removal of the aforesaid from the fermentation tank, can be precisely acquired by way of the weighing device associated with the vacuum pressure pumping system, and can thus be automatically controlled. It is thus possible to let this process flow in a controlled manner and in this way achieve a situation in which a combined heat and power unit operated by means of the biogas produced is always operated as far as possible at full load.

It is then possible, in the mixing tank, for hydrolysis of the fermentation material to take place preferably already during the fermentation processes. In the mixing tank already a largely homogeneous, in particular thick-flowing, mixture is produced so that homogeneous new fermentation material with as low a water content as possible is fed to the main tank at a defined point in time. In this way a more uniform fermentation process in the main tank can be achieved.

Basically, according to a modification of the above-mentioned principle, in the context of the invention the mixing tank can also be partially annular in shape and can partially extend circumferentially around the fermentation tank. It is not necessary for the mixing tank to be annular in shape, nor is it mandatory for it to extend around the fermentation tank; instead, according to a variant of the invention, said mixing tank can also comprise some other geometric form and can be accommodated at some other location so as to be separate from the fermentation tank.

Mixing in the mixing tank can take place by means of agitators which are, for example, arranged laterally on the wall of said mixing tank. In this arrangement it does not matter if foreign objects, for example stones, reach the mixing tank together with the fermentation material, because these foreign objects fall to the bottom of the mixing tank; and preferably a vacuum pressure pumping system can be used for conveying the fermentation material out of the mixing tank and into the fermentation tank. In contrast to conventional pumping systems, in this arrangement the fermentation material does not establish contact with mechanical components of a pump, which components would be damaged by such foreign objects. In the fermentation tank itself, conventional agitators can be used.

The fresh material to be treated can be circulated multiple times in the annular mixing tank until the desired degree of mixing and, if applicable, in the case of hydrolysis the desired breakdown state has/have been reached. This provides improved control over the quantity of the newly admixed material to be treated.

Preferably, the width of the mixing tank that extends annularly around the fermentation tank is narrower than the radius of the cylindrical part of the fermentation tank, wherein preferably the width of the mixing tank is less than half the radius of the cylindrical part of the fermentation tank. Preferably, the height of the mixing tank is also lower than the height of the cylindrical part of the fermentation tank, wherein the mixing tank is preferably arranged in the bottom region of the fermentation tank, in particular below the ground level surrounding the tank. The quantity of the material to be treated in the mixing tank is thus preferably only a fraction of the quantity of the fermentation material in the fermentation tank.

In the mixing tank a thick-flowing mash can be mixed from the freshly fed-in organic substances, for example comprising dry matter of 20% to 35%. However, this strongly depends on the substances that have been admixed. Apart from the usual fermentable organic substances used in the production of biogas (in particular liquid manure) suitable starting substances or admixtures include, for example, maize, grass silage, crushed grain, bread waste and the like, or also liquids such as, for example, glycerine.

A further advantage of the annular mixing tank results from its geometry. In contrast to an angular tank, an annular tank does not have any dead zones, and consequently it is considerably easier to achieve a uniform mixture.

In the context of the present invention the principle can also be applied to silos or tanks for storing organic material, around which silos or tanks an annular or only partially annular mixing tank is arranged. In this case the fermentation processes could, for example, take place in a further separate tank. However, from the point of view of process technology this only makes sense if the actual fermentation tank is not too far away from the mixing tank so that excessively long pumping distances are avoided.

According to an improvement of the invention, preferably a vacuum pressure pumping system is used for conveying the material to be treated out of the mixing tank and into the fermentation tank. This pumping system is particularly suitable for pumping thick-flowing tenacious media. Even if said media contain foreign objects such as stones or the like, this does not matter. In this arrangement, the material to be treated is in the first instance aspirated by negative pressure, for example into a tank, and then pressed out of said tank by means of compressed air and into the respective container. In contrast to the use of conventional pumps with moving mechanical components, in this arrangement there is no danger of the aforesaid being damaged by foreign objects. Accordingly, the material to be treated is not pumped by a conventional mechanical pump directly from the mixing tank to the fermentation tank, but instead is first aspirated from the mixing tank and then pressed into the fermentation tank.

For example, in each case only relatively small quantities (relative to the overall quantity of the material to be treated, which material can be treated in the plant) can be pumped into the mixing tank. It is advantageous if the mixing tank is at all times at least partially full when the plant is operating. If some of the mixed material to be treated is pumped out of the mixing tank into the fermentation tank, fresh material to be treated can be pumped into the mixing tank in order to keep the fill level (as a rule partial filling only) in the mixing tank approximately constant. If it is detected that no homogeneous mixture is yet present in the mixing tank, the residence time in the mixing tank can be extended. In the case of anaerobic fermentation of fermentable organic media, hydrolysis occurs in the annular mixing tank if the residence time in the mixing tank is, for example, one day or several days. The largely homogeneous, already hydrolysed, fermentation material can then be pumped to the main fermentation tank for further fermentation.

It is particularly advantageous if according to a preferred improvement of the invention fresh fermentation material to be placed into the mixing tank is first pumped (aspirated) out of a storage tank into a tank (of the vacuum pressure pumping system) and only thereafter is pumped (pressed) out of the tank into the mixing tank, and/or if pre-treated fermentation material taken out of the mixing tank is first pumped into a tank and then into the fermentation tank, and/or if fermented fermentation material is first pumped out of the fermentation tank into a tank and is then pumped out of the tank into a final storage place (storage tank). In other words the fresh fermentation material is thus not pumped directly into the mixing tank, but first into the tank of the vacuum pressure pumping system. The pump used for this purpose is situated outside this tank and outside the pipe path used for conveying the fermentation material. The pump itself does thus not establish contact with the fermentation material. For drawing the medium into the tank of the vacuum pressure pumping system by suction, and for pressing the medium out of the aforesaid, the same line system may be used.

Thus, preferably, fermentation material is not transferred from the mixing container directly to the fermentation tank, but always by means of the interconnected tank. If in this process stones or other foreign objects are also drawn in by suction, this does not matter.

If according to a preferred improvement of the invention the tank of the vacuum pressure pumping system is arranged both in close proximity to the mixing tank and in close proximity to the fermentation tank, there is a further advantage in that the same tank and the same vacuum pressure pumping system can be used for pumping fresh fermentation material into the mixing tank, for emptying the mixing tank, and for pumping into the fermentation tank the fermentation material that has already been pre-treated in the mixing tank, and for removing from the fermentation tank fully fermented fermentation material (fermentation residue). The pumping distances are thus very short, which is advantageous in the case of high dynamic viscosity of the fermentation material. In this context it is, for example, also advantageous if the mixing container is arranged below the ground level surrounding the fermentation tank, so that the tank can be arranged beside the fermentation tank and above the lower-lying mixing tank and is thus in close proximity to both tanks.

The object of the present invention further relates to apparatus for use in a method of the type described above, which apparatus is characterised in that the fermentation tank is of cylindrical design, and in that the mixing tank extends in an annular shape or in a partly annular shape around the cylindrical fermentation tank, wherein both the respective spaces of the two tanks in which the thick-flowing fermentation medium is located, and the respective gas spaces of the tanks above the fermentation medium are completely separate. The above-mentioned at least partly cylindrical tank can, for example, be a round silo, a basin or a tank that contains organic substances. The mixing tank extends annularly, preferably concentrically to the axis of the tank around said tank. The mixing tank can, for example, comprise a rectangular cross section.

The object of the present invention, in particular, relates to apparatus for treating fermentable organic substances by means of anaerobic fermentation in a fermentation tank comprising devices for mixing the fermentation material, with at least some of said devices being arranged outside the fermentation tank, wherein said apparatus is characterised in that it comprises an annular or partly-annular mixing tank to which the fresh fermentation material is fed, which mixing tank extends in an annular shape or in a partly annular shape circumferentially around the fermentation tank, wherein a vacuum pressure pumping system is provided as a device for conveying the fermentation material to the mixing tank, wherein this pumping system is preferably also used to convey the fermentation material from the mixing tank to the fermentation tank. This fermentation tank is, in particular, a tank for the production of biogas from fermentable organic substances. In the mixing tank, in particular, an energy-rich substance mash that is as thick-flowing as possible is produced which can then be pumped into the fermentation tank, preferably by means of compressed air. However, in this arrangement, this pumping process takes place in such a manner that the fermentation material is first pumped out of the mixing tank and only thereafter is pumped into the fermentation tank. There is thus preferably no direct connection line between the mixing tank and the fermentation tank. This makes it possible, on the one hand, to control, for example to weigh or enrich, the quantity removed from the mixing tank, and on the other hand, it is not mandatory for the quantity of fermentation material removed from the mixing tank to be conveyed at the same volume to the fermentation tank. Furthermore, by using the vacuum pressure pumping system, the mixture does not come into direct contact with mechanical components of a pump, and in an advantageous manner comparatively short pumping distances result.

The mixing tank according to the invention thus preferably forms an annular mixing device that extends around a fermentation tank or a silo, in which mixing device the fermentation process can already start. In particular in the case of anaerobic fermentation by means of bacteria for the production of biogas, hydrolysis can take place already in the mixing device. This depends, among other things, on the type of biomass and on the residence time in the mixing tank. If applicable, further decomposition steps and fermentation steps can already take place in the mixing tank, for example acidogenesis/acetogenesis, wherein organic components of the biomass are transformed by anaerobic bacteria to form acids, and consequently the pH-value in the mixture decreases.

The annular mixing tank can, for example, be of a shorter height than the fermentation tank, and the mixing tank can, for example, also be arranged below the ground level of the surroundings (in other words quasi-embedded in the ground). In this case it is a type of hydrolysis cellar that circumferentially extends around the fermentation tank, whose lowest region can also, for example, be set into the ground. This can be advantageous to keep the fermentation material in the mixing tank cooler, or to maintain a more even temperature irrespective of the season. In the production of biogas, the fermentation tank itself is mostly only cylindrical in its lower partial region, while adjacent towards the top it comprises a dome or a bell in which the biogas collects.

Preferably, the apparatus furthermore comprises a vacuum pressure pumping system for conveying the organic substances or the fermentation material, with the vacuum pressure pumping system comprising a tank to which a vacuum can be applied in order to draw in, by suction, the organic substances or the fermentation material, which tank comprises a closing device, wherein the organic substances or the fermentation material can subsequently be removed from the tank by means of compressed air. Preferably, this vacuum pressure pumping system comprises at least one pump, arranged outside the tank, and at least one line for air, which line leads from the pump to the tank, in order to pressurise the tank with compressed air or to apply negative pressure to the tank. This makes it possible to also draw into the tank, by suction, a thick-flowing medium that may contain foreign objects, and subsequently remove said medium by pressurising the tank with compressed air.

Particularly preferably, the mixing tank comprises a volume that is separate from the fermentation tank, wherein at least one line for fermentation material leads from the mixing tank to the tank in order to charge or discharge the mixing tank, and wherein at least one line extends from the tank to the fermentation tank in order to charge or discharge the fermentation tank, so that, for example, with only one tank both the mixing tank and the fermentation tank can be charged or discharged. However, it is not provided for the fermentation material to be able to flow, by way of an opening in the mixing tank or by way of a line, from the mixing tank directly into the fermentation tank.

The characteristics mentioned in the subordinate claims relate to preferred improvements of the solution according to the invention. Further advantages of the invention are stated in the following detailed description.

### BRIEF DESCRIPTION OF THE DRAWINGS

Below, the present invention is explained in more detail by means of exemplary embodiments with reference to the enclosed drawings. The following are shown:

FIG. 1 a top view of a plant according to the invention with a fermentation tank and an annular mixing tank;

FIG. 2 a longitudinal section of the plant shown in FIG. 1;

FIG. 3 a partly diagrammatical detailed view of a vacuum pressure pumping system according to the invention for conveying the fermentation material in five different operating phases;

FIG. 4 a diagrammatic view of a plant according to the invention in six different operating phases.

### DETAILED DESCRIPTION OF THE PREFERRED EMBODIMENT

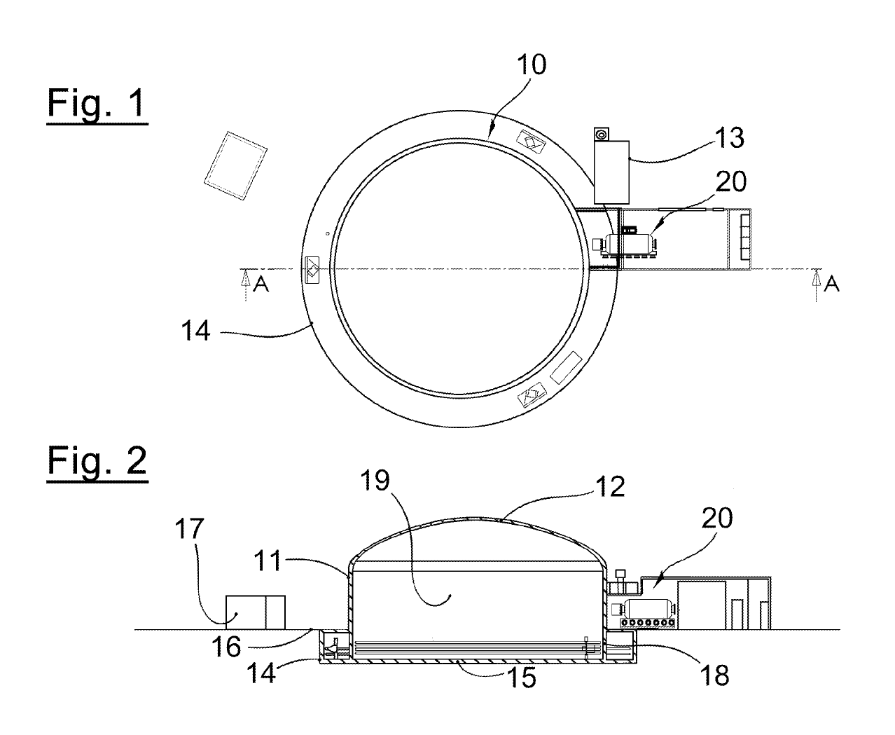

In the first instance, reference is made to FIGS. 1 and 2, and with reference to the aforesaid a possible embodiment of the method according to the invention is explained in more detail. The illustrations show a fermentation tank 10 which in its lower region 11 is of a cylindrical design and which in its upper region comprises a curved dome 12 that is closed at the top so that underneath this dome 12 the produced biogas can collect. The biogas can then be led away and can be used for generating electricity, for example in an adjacent combined heat and power unit 13. As shown, in particular in FIG. 2, the fermentation tank 10 in the lower part of the cylindrical region 11 is on the outside concentrically enclosed by an annular mixing tank 14 that is rectangular in cross section wherein the latter is positioned in such a manner that on the bottom it closes off so as to be horizontally flush with the bottom 15 of the fermentation tank 10, while the mixing tank 14 at the top finishes off approximately at the height of the ground surface 16 of the terrain surrounding the plant. The mixing tank 14 is thus quasi-embedded in the ground and in total comprises a significantly lower overall height than does the fermentation tank. Correspondingly, the volumetric capacity of the mixing tank 14 is several times less than that of the fermentation tank 10.

As a rule, a biological air filter 17 is used to remove the burden on the environment as a result of unpleasant odours. On its radial inside the annular mixing tank 14 is separated from the interior space 19 of the fermentation tank 10 by a separating wall 18 which is, for example, flush with the cylindrical region 11. A vacuum pressure pumping system 20 is used for filling the mixing tank 14 and the fermentation tank 10. This vacuum pressure pumping system 20 can be arranged at the level of the ground surface 16 so that said vacuum pressure pumping system 20 is situated above the mixing tank 14. Furthermore, the vacuum pressure pumping system is preferably arranged in close proximity to the fermentation tank 10 and to the mixing tank 14 so as to ensure short pumping distances.

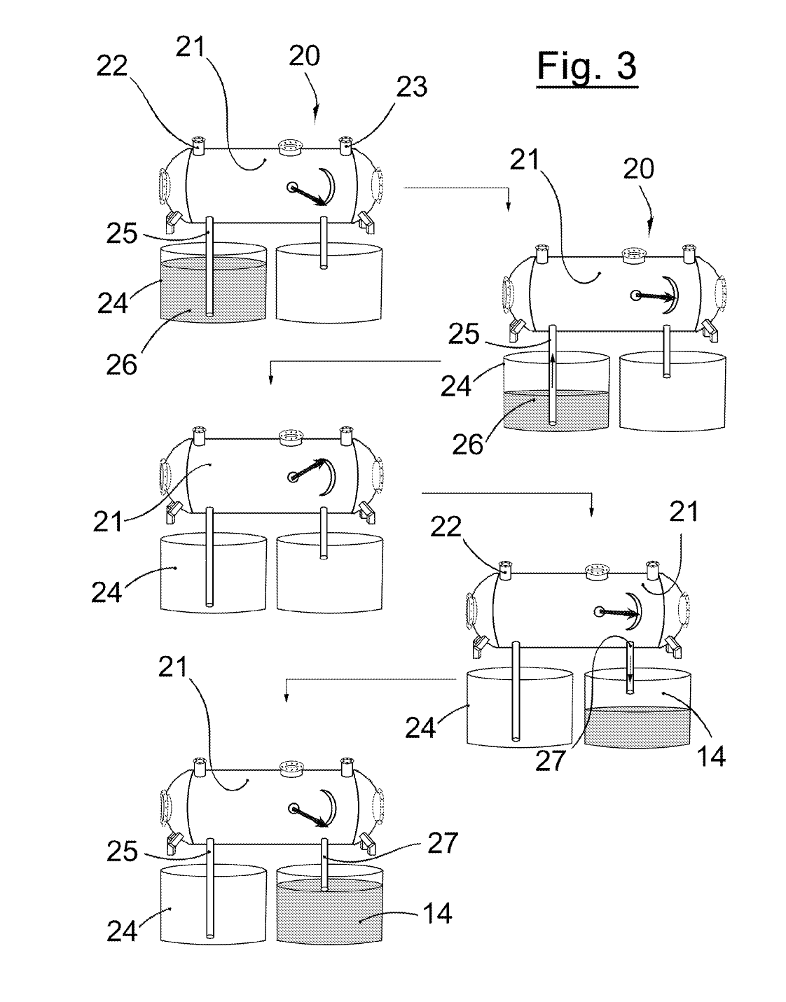

Below, the function of the vacuum pressure pumping system 20 is explained in more detail with reference to FIG. 3. This pumping system comprises a tank 21 with connections 22, 23 for one or several pumps so that a vacuum or compressed air can be applied to the interior of the tank. In the first diagram, top left of FIG. 3, this tank 21 is empty. From a diagrammatically shown storage tank 24, fermentation material 26 can be drawn into the tank 21 by way of suction via a line 25, provided that previously negative pressure of, for example 0.5 bar, has been applied to the interior space of the tank 21 so that said tank 21 can consequently be filled up to a predetermined fill level that is, for example, predetermined by means of a float or the like. As an alternative it is also possible to check the fill level, for example by way of weighing cells or similar devices (in other words the tank as well as its contents are weighed). The second diagram, top right, shows the manner in which the tank 20 is gradually filled with fermentation material 26 from the storage tank 24 by way of the line 25.

The third diagram of FIG. 3, middle left, shows the state after the tank 21 has been filled, wherein the stored fermentation material was drawn by suction from the storage tank 24 to the tank 21. After this the process of discharging the tank 21 can commence, in which process, as shown in the fourth diagram of FIG. 3, bottom right, overpressure of, for example, 1.5 bar is applied to the interior of the tank 21, for example by way of the connection 22, and consequently the fermentation material can be pumped out of the tank 21, by way of a further line 27, into a further tank, in the illustration shown diagrammatically only, for example into the annular mixing tank 14 as shown in FIG. 2.

In the fifth diagram of FIG. 3, bottom left, the tank 21 is empty, and the fermentation material is located in the mixing tank 14. Thus the fermentation material can be transferred by way of the tank 21 out of the storage tank 24 into the mixing tank 14 without the pump that generates negative pressure or overpressure in the tank, or without the line system between the pump and the tank establishing any contact whatsoever with the fermentation material. Instead, the fermentation material is drawn by negative pressure from one tank 24 to the other tank 21, and by overpressure is subsequently pressed out of the tank 21 into the other tank 14. It is thus also not possible for the pump to be negatively affected by foreign objects in the fermentation material.

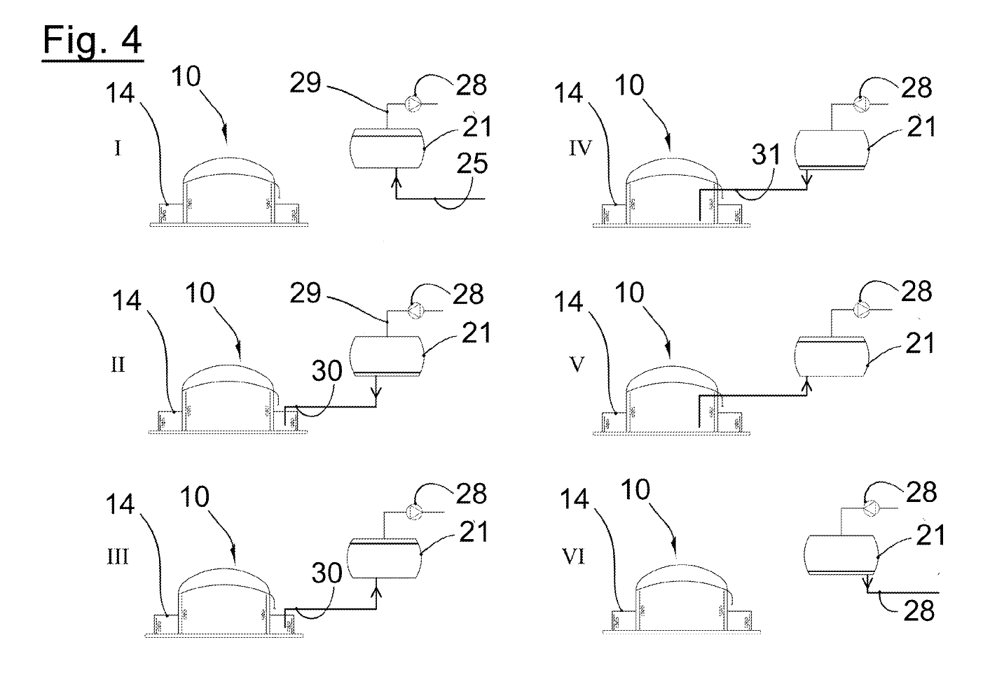

Below, the mode of operation of the plant according to the invention is explained in more detail with reference to the six schematic diagrams of FIG. 4. FIG. 4 in the first diagram, top left, shows the fermentation tank 10 and the mixing tank 14. Also shown is the previously described tank 21, into which, by way of the supply line 25, from a reservoir (not shown) of fermentation material, the fermentation material is drawn by suction due to the negative pressure in the tank 21. The pump 28 that removes air from the tank 21 by suction by way of the line 29 is also diagrammatically shown. In the illustration the tank 21 is already largely full of fermentation material.

The second diagram in FIG. 4, middle left, shows the process of pumping the fermentation material out of the tank 21 by way of a supply line 30 into the mixing tank 14. To this effect, pressure is applied to the interior space of the tank 21, wherein said pressure is generated by the pump 28, which in this case delivers compressed air 9 to the tank 21 by way of the line 29, instead of drawing in by suction. In this manner in the first instance the mixing tank 14 can be filled with fresh fermentation material that remains in the mixing tank for some time, for example one day, two days or longer.

The third diagram in FIG. 4, bottom left, shows the process of drawing fermentation material by suction out of the mixing tank 14 and into the tank 21, wherein in this case the pump 28 uses suction again so that negative pressure arises in the tank 21, and the fermentation material is drawn into the tank again by suction, by way of the line 30, after having stayed in the mixing tank 14 for the duration of the hydrolysis phase.

The fourth diagram in FIG. 4, top right, shows the fermentation material being pumped out of the tank 21 and into the fermentation tank 10 by way of the line 31 in that, by means of the pump 28, compressed air is applied to said fermentation tank 10. In this arrangement it is possible, at least in part, to use the same line system for the pumping process, or the tank 21 can have several outlets for fermentation material, as indicated in the schematic drawing.

After the fermentation material has remained in the fermentation tank 10 for the envisaged period of time, and after fermentation has been completed, as shown in the fifth diagram of FIG. 4, middle right, the fermentation material is pumped out of the fermentation tank 10 and back into the tank 21 in that the pump 28 again applies suction so that negative pressure arises in the tank 21.

Finally, in a concluding step, according to the sixth diagram of FIG. 4, bottom right, by application of pressure to the tank 21 the fermentation material (fermentation residue) is pumped out of the aforesaid and into a final storage place by way of a line 32. In this arrangement, if applicable, in part the same line system can be used that is also used for the supply of fresh fermentation material to the tank (see FIG. 4 first diagram). However, the tank can also comprise a corresponding number of different outlets. It is understood that corresponding valves are provided on the inlets/supply lines and outlets/discharge lines or on connection pieces of the tank in order to selectively support the various above-mentioned functions during charging or discharging the tank, and to open or close the respectively needed inlets/outlets and/or lines.

During operation of a biogas plant with a mixing tank 14 according to the present invention, as a rule the aim is to produce as thick-flowing and energy-rich a mash as possible in the mixing phase/hydrolysis phase. In the method according to the invention, preferably, in each case smaller portions of the fermentation products are pumped out of the mixing tank 14 and into the tank 21, and subsequently out of the tank into the fermentation tank 10. Preferably, the mixing tank is always kept partially full. For example, if this mixing tank has a content of 400 m3, for example every day a quantity of 100 m3 is pumped out of the mixing tank, by way of the tank, into the fermentation tank 10. A couple of times a week, fresh fermentation material is pumped from a reservoir, first into the tank 21 and then from the aforesaid into the mixing tank 14. Preferably, the process is managed in such a manner that the mixing tank 14 always remains partly full, for example always approximately half full. To achieve hydrolysis, the residence time of the fermentation material in the mixing tank is preferably approximately two days or longer. In the hydrolysis phase (as is the case in the subsequent fermentation phase in the main tank) it is important to ensure that the process takes place so as to exclude oxygen. The method according to the invention also provides an advantage in that during the conveyance of fresh fermentation material into the mixing tank 14 the fermentation material present therein is displaced and, due to the annular shape of the mixing tank, the fermentation material is quasi-conveyed on a circular path in said mixing tank 14. Consequently the added fresh fermentation material mixes in the inlet region with fermentation material that has already had an extended residence time in the mixing tank.

### LIST OF REFERENCE NUMERALS

10 Fermentation tank
11 Lower cylindrical region
12 Dome
13 Combined heat and power unit
14 Mixing tank
15 Bottom of the fermentation tank
16 Ground surface of the surroundings
   17 Biological air filter
   18 Separating wall
   19 Interior space
20 Vacuum pressure pumping system
21 Tank
22 Connection
23 Connection (connection piece)
24 Storage tank
25 Line
26 Fermentation material
27 Line
28 Pump
29 Line
30 Line
31 Line
32 Line

1. A method for treating fermentable organic substances, comprising the anaerobic fermentation thereof in at least one fermentation tank, and equipment to mix the fermentation material, wherein the fresh fermentation material fed, in the first instance, is fed to a mixing tank (14) in which the fermentation material is mixed, chopped up if necessary and mixed into a thick-flowing mash, and only thereafter is this mixture fed to a fermentation tank for further fermentation, characterised in that after a certain residence time the fermentation material is conveyed out of the mixing tank and fed to the fermentation tank (10) by means of a vacuum pressure pumping system (20).

2. The method according to claim 1, characterised in that the mixing tank (14) and the fermentation tank (10) are separate tanks, with no immediate direct fluid connection from the mixing tank to the fermentation tank being provided.

3. The method according to claim 1, characterised in that the fermentation material is hydrolysed in the annular mixing tank before it is pumped into the fermentation tank for further fermentation.

4. The method according to claim 1, characterised in that by means of the vacuum pressure pumping system (20) the fermentation material is also conveyed into the mixing tank (14) and/or within the mixing tank.

5. The method according to claim 1, characterised in that in the mixing tank the fermentation material is mixed to form a thick-flowing mash comprising, preferably, 20% to 35% dry matter, before said fermentation material is pumped into the fermentation tank.

6. The method according to claim 1, characterised in that in each case only a fraction of the mixed fermentation material situated in the annular mixing tank is removed from said mixing tank and is pumped into the fermentation tank.

7. The method according claim 1, characterised in that during operation of the plant the mixing tank is always partly full of fermentation material.

8. The method according to claim 1, characterised in that fresh fermentation material to be placed into the mixing tank (14) is first pumped out of a storage device into a tank (21) and only thereafter is pumped out of the tank into the mixing tank, and/or in that pre-treated fermentation material taken out of the mixing tank is first pumped into a tank (21) and is then pumped into the fermentation tank (10), and/or in that fermented fermentation material is first pumped out of the fermentation tank into a tank (21) and is then pumped out of the tank into a final storage place.

9. An apparatus for use in a method according to claim 1, wherein the fermentation tank (10) is of cylindrical design, and the mixing tank extends in an annular shape or in a partly annular shape around the cylindrical fermentation tank (10), wherein the respective spaces of the two tanks in which the thick-flowing fermentation medium is located are completely separate, characterised in that also the respective gas spaces of the tanks above the fermentation medium are completely separate.

10. The apparatus according to claim 9, characterised in that the width of the mixing tank (14) that extends annularly around the fermentation tank (10) is narrower than the radius of the cylindrical part of the fermentation tank, wherein the width of the mixing tank is less than half the radius of the cylindrical part of the fermentation tank, and the height of the mixing tank (14) is lower than the height of the cylindrical part of the fermentation tank (10).

11. The apparatus according to claim 10, characterised in that the mixing tank is arranged in the bottom region of the fermentation tank, and below the ground level (16) surrounding the tank.

12. The apparatus according to claim 9, characterised in that a vacuum pressure pumping system (20) is provided for conveying the organic substances or the fermentation material, with the vacuum pressure pumping system (20) comprising a tank (21) to which a vacuum can be applied in order to draw in, by suction, the organic substances or the fermentation material, which tank (21) comprises a closing device, wherein the organic substances or the fermentation material can subsequently be removed from the tank by means of compressed air.

13. The apparatus according to claim 12, characterised in that the mixing tank (14) comprises a volume that is separate from the fermentation tank (10), in that at least one line (30) for fermentation material extends from the mixing tank (14) to the tank (21) in order to charge or discharge the mixing tank, and in that at least one line (31) extends from the tank (21) to the fermentation tank (10) in order to charge or discharge the fermentation tank.

14. The apparatus according to claim 12, characterised in that the vacuum pressure pumping system (20) comprises at least one pump (28), arranged outside the tank (21), and at least one line (29) for air, which line leads from the pump to the tank, in order to pressurise the tank with compressed air or to apply negative pressure to the tank.

15. The apparatus according to claim 9, characterised in that the tank (21) is positioned on a weighing device by means of which the weight of the respective substance quantity placed in the tank and/or of the substance quantity removed from said tank are/is acquirable.
